(19) United States
(12) Patent Application Publication
(10) Pub. No.: US 2005/0101032 A1
(43) Pub. Date: May 12, 2005

(54) Assay device, composition, and method of optimizing assay sensitivity

(75) Inventors: Joel M. Blatt, Mountain View, CA (US); Wilma M. Mangan, Santa Clara, CA (US)

Correspondence Address: BURNS DOANE SWECKER & MATHIS L L P, POST OFFICE BOX 1404, ALEXANDRIA, VA, 22313-1404, US

(73) Assignee: Metrika, Inc., Sunnyvale, CA (US)

(21) Appl. No.: 10/927,637

(22) Filed: Aug. 27, 2004

Related U.S. Application Data
(63) Continuation of application No. 08/796,780, filed on Feb. 10, 1997, now abandoned.

(51) Int. Cl. G01N 33/563; G01N 33/543

(52) U.S. Cl. 436/518

(57) ABSTRACT

The present invention provides an assay composition for producing a physically detectable change upon contact with a sample which correlates with the amount of selected analyte in the sample. The composition includes a support matrix pervious to optical radiation and a chemical reagent yielding a physically detectable change which correlates with the amount of selected analyte in the sample. The support matrix has at least one detection zone for detecting the physical change with a cross-sectional area and depth profile. The composition includes an opacifier present in an amount sufficient to increase the resolution of the physically detectable change. The opacifier is distributed uniformly across the cross-sectional area of the detection zone and is distributed through at least a portion of the depth profile of the support matrix within the detection zone. The chemical reagent is substantially immobilized relative to the opacifier when detecting the physical change. The assay composition can be supported on a transport matrix and used in an assay device. The present invention determines the level of a selected analyte in a sample by optimizing a physically detectable change in an assay composition which correlates with the amount of selected analyte when contacted with the sample. The present invention optimizes the assay results of a selected analyte in a sample-exposed assay composition by increasing the reflection of optical radiation detecting a physical change of the sample-exposed assay composition to be within the range of optimal assay resolution.

### RELATED APPLICATIONS

The subject matter of this application is related to a disposable single-use digital electronic instrument that is entirely self-contained, including all chemistry reagents, as disclosed in U.S. Pat. No. 5,580,794 entitled “Disposable Electronic Assay Device” by Michael P. Allen; U.S. application Ser. No. 08/512,844 entitled “Dry Reagent Particle Assay And Device Having Multiple Test Zones And Method Therefor” filed Aug. 9, 1995 by Joel M. Blatt and Michael P. Allen; U.S. application Ser. No. 08/642,228 filed Apr. 30, 1996 by Raymond T. Hebert et al.; and U.S. application Ser. No. 08/645,453 entitled “Method And Device Producing A Predetermined Distribution Of Detectable Change In Assays” filed May 13, 1996 by Joel M. Blatt et al. The above applications have the same assignee as the present invention and are incorporated herein by reference in their entirety.

### FIELD OF THE INVENTION

The present invention relates to a device, composition, and method which optimizes the resolution of detecting a physical change on the surface of a sample-exposed analytical chemistry strip in a diagnostic device which displays medical information.

### BACKGROUND OF THE INVENTION

In the past, immunoassays were developed for the quantitative and qualitative determination of a wide variety of compounds in a laboratory setting using detailed procedures and expensive instrumentation. Recent developments in immuno-diagnostics have resulted in a movement toward more simple approaches to the rapid analysis of clinical samples. The development of solid phase bound reagents has eliminated the need for precipitation in the separation of bound reagents from free reagents. Further advancements in solid phase immunochemistry have resulted in non-instrumented dry reagent strip immunoassays. This configuration allows for the visual qualitative or semi-quantitative determination of analytes in patient samples without the use of an instrument. To improve the accuracy and reliability of assay results, several qualitative and quantitative diagnostic tests have been developed in the clinical field utilizing a reflectometer for measuring optical radiation reflected from a test element such as a zone of detection on an analytical chemistry strip.

Whether the reflected optical radiation is detected visually or with the assistance of a reflectometer, problems often arise obtaining accurate and precise measurements of the diffusely reflected optical radiation from the surface of the assay strip within the sampling area or detection zone. One of the causes is that the physically detectable change which is being measured in the test area does not occur within the most sensitive or optimal range of the analytical assay. Assay results detected outside of the optimal range are usually not as accurate by comparison.

There is a need to achieve greater resolution to the physically detectable change across a detection zone using an indicator or other signal producing reagent. It is also desirable to optimize detection, whether visually or by using an instrument, of a physical change in a detection zone of an assay strip.

Thus, a need exists in the field of diagnostics for a device, composition, and method which modifies the reflection of optical radiation for detecting a physical change on the surface of a sample-exposed analytical chemistry strip to be within the range of optimal assay resolution. The optimization of assay resolution should be sufficiently inexpensive, timely, efficient, durable, and reliable for use in a diagnostic device which permits point-of-care use by untrained individuals in locations such as the home, sites of medical emergencies, or locations other than a clinic.

### SUMMARY OF THE INVENTION

The present invention provides an assay composition for producing a physically detectable change upon contact with a sample which correlates with the amount of selected analyte in the sample. The composition includes a support matrix pervious to optical radiation and a chemical reagent yielding a physically detectable change which correlates with the amount of selected analyte in the sample. The support matrix has at least one detection zone for detecting the physical change. The detection zone having a cross-sectional area and a depth profile extending into the support matrix. The composition includes an opacifier present in an amount sufficient to increase the resolution of the physically detectable change. The opacifier is distributed uniformly across the cross-sectional area of the detection zone and is distributed through at least a portion of the depth profile of the support matrix within the detection zone. The chemical reagent is substantially immobilized relative to the opacifier when detecting the physical change.

Another aspect of the present invention is a transport matrix which produces a physically detectable change in a detection zone which correlates with the amount of selected analyte in a sample. The matrix includes a detection zone having a chemical reagent yielding a physically detectable change which correlates with the amount of selected analyte in the sample and an opacifier present in an amount sufficient to increase the resolution of detecting the physical change. The detection zone has a cross-sectional area and a depth profile extending into the matrix. The opacifier is distributed uniformly across the cross-sectional area of the detection zone and being distributed through at least a portion of the depth profile of the matrix within the detection zone. The opacifier is substantially immobilized relative to the chemical reagent in the detection zone when detecting the physical change.

The present invention also provides a diagnostic device for determining the presence of a selected analyte in a sample. The device includes a housing having an exterior surface and sealing an interior area. A receptor is configured to receive the sample containing an analyte selected for determining its presence. The receptor is located on the exterior surface of the housing. At least one transport matrix of the type described above is provided for reacting the sample with a chemical reagent to yield a physically detectable change in a detection zone which correlates with the amount of selected analyte in the sample.

The present invention also provides a method for determining the level of a selected analyte in a sample. The method includes optimizing a physically detectable change in an assay composition which correlates with the amount of selected analyte when contacted with the sample.

Another method provided by the present invention optimizes the assay results of a selected analyte in a sample-exposed assay composition. The method includes increasing the reflection of optical radiation detecting a physical change of the sample-exposed assay composition to be within the range of optimal assay resolution.

The advantages, embodiments, variations and the like will be apparent to those skilled-in-the-art from the present specification taken with the accompanying drawings and appended claims.

### BRIEF DESCRIPTION OF THE DRAWINGS

In the drawings, which comprise a portion of this disclosure:

FIG. 1 is a partial top plan view of a diagnostic device having a portion cut-away to view the illumination and detection optics of the present invention;

FIG. 2 is a partial cross-sectional view of the diagnostic device illustrated in FIG. 1 along the lines 2-2;

FIG. 3 is a top plan view of a non-instrumented diagnostic device using the present invention;

FIG. 4 is an isolated view of a transport matrix having two detection zones utilizing the present invention;

FIG. 5 is a graph of error in K/S (%) versus percentage change in reflectance (% R) generally illustrating measurement error as a function of reflectance and analog/digital resolution of an instrument detecting the physical change;

FIG. 6 is a side view of one embodiment of an assay strip suitable for use in a general chemistry assay such as analyzing samples containing sarcosine;

FIG. 7 is a top plan view of the assay strip in FIG. 6;

FIG. 8 is a graph of the K/S value for four levels of titanium dioxide (5%, 10%, 15%, and 20% w/v TiO₂) versus different concentrations of sarcosine (mM) in a sample; and

FIG. 9 is a graph of the K/S value for three levels of titanium dioxide (10%, 15%, and 20% w/v TiO₂) versus different concentrations of sarcosine (mM) in a sample.

### DESCRIPTION OF THE PREFERRED EMBODIMENTS

The present invention may be utilized in either instrumented or non-instrumented assay devices. Examples of instrumented devices include the disposable single- and multiple-use digital electronic instruments and assay devices described in detail in the above-identified related applications previously incorporated by reference. The present invention provides for more accurate measurement, by either visual observation or instrumentation, of a physically detectable change corresponding to the amount of the selected analyte in one or more detection zones of an assay.

Generally, the present invention optimizes the resolution of an assay composition which produces a physically detectable change upon contact with a sample which correlates with the amount of selected analyte in the sample. The assay resolution is optimized by increasing the amount of optical radiation reflected from the assay composition to fall within the most sensitive range of reflectance for the concentration range of clinical interest of the selected analyte. The present invention positions elements within the assay composition which scatter the optical radiation directed into assay composition through its surface. The scattering elements increase the amount of optical radiation which is reflected out of the assay composition back through the surface the optical radiation entered. The reflected optical radiation is then detected either visually or with the assistance of an optically sensitive instrument like a reflectometer.

One type of scattering element suitable for use as the present invention is an opacifier. The term opacifier as used herein is defined as an element added to a material to make it opaque which makes the material less penetrable by light, but still reflects light. The opacifier is present in an amount sufficient to increase the resolution of detecting the physical change by scattering the optical radiation directed into the assay composition and increasing the reflectance of the optical radiation to where it is detected.

Since the opacifier scatters light, it is preferably distributed within at least a portion of the depth profile of the assay composition. Distributing the opacifier on a surface of the assay composition, either closest or furthest from the point which the optical radiation enters the assay composition, produces a negligible increase in assay resolution.

Distributing the opacifier only at the surface closest to the entrance of the optical radiation, without penetration into the depth of the assay composition, will immediately reflect optical radiation back to the observer without scattering it through the assay composition itself. As a result, assay resolution decreases because less light penetrates into the assay composition. The only increase in scattered light occurs at the assay composition surface which results from the light hitting the opacifier on the surface and not within the assay composition. Most of the incident light scattering back to the observer does not pass through the assay composition.

Positioning the opacifier at the opposite surface of the assay composition, furthest from where the optical radiation enters, determines the total amount of optical radiation reflected, but does not modulate the reflected optical radiation. The result is a negligible increase in the resolution of the assay.

In order to promote the scattering effect of the opacifier, it is distributed through at least a portion of the depth profile of the assay composition within a detection zone for detecting the physical change. Increasing the number of scattering centers shortens the mean path length of the optical radiation between striking different scattering centers within the assay composition and shortens the path length between the points of entry and exit for the optical radiation. Preferably, the opacifier is distributed uniformly through the entire depth profile of the assay composition in the detection zone when the reflected optical radiation is detected. The opacifier is also distributed uniformly across the cross-sectional area of the detection zone to promote reliable and reproducible detection of the physical change.

The assay composition includes a chemical reagent or an indicator which are defined herein to yield a physically detectable change which correlates with the amount of selected analyte in the sample. The indicator generally scatters a negligible amount of the optical radiation directed into the assay composition. The opacifier modifies the physical change, such as color intensity, yielded by the indicator by increasing the ratio of scattered light over absorbed light.

The assay composition also includes a support matrix pervious to the optical radiation. The support matrix includes at least one detection zone for detecting the physical change. The detection zone has a cross-sectional area and a depth profile extending into the matrix. The support matrix defines the physical distance and relation of the chemical reagent and the opacifier to one another within the detection zone. It is preferred that the physical relationship between the opacifier and the chemical reagent is substantially immobilized at the time the reflected optical radiation from the physical change is detected. A material suitable for use as the support matrix in the assay composition includes, but is not limited to, conventional water-based gels like polyvinyl alcohol, polyethylene glycol, or gelatin and translucent materials which include porous materials of various opacities such as microporous membranes.

An opacifier suitable for use in the present invention should be inert and stable in contact with biologically active molecules such as the reaction of the reagent and sample analyte. The opacifier should also be finely dispersible with a small particle size. Ease of uniformly dispersing the opacifier is desirable. The opacifier should also have a refractive index greater than that of water.

Preferably, the opacifier has an index of refraction in the range of about 2.5 to about 3. The most preferred particle size of the opacifier is in the range of about 0.2 μm to about 0.4 μm with a preferred transport matrix pore size of about 1 μm to about 8 μm, although a pore size of about 0.451 μm to 10 μm is suitable for light in the visible portion of the electromagnetic spectrum. The optimal particle size is dependent on the scattering efficiency of the centers and the wavelight of light. The optimal particle size will increase for light in the near infrared region, and decrease for light in the far ultraviolet region of the spectrum. Preferably, the opacifier has a surface area in the amount greater than about 10 m²/g.

A preferred material for use as the opacifier in the present invention is titanium dioxide, particularly in the rutile form. Other suitable materials include barium sulfate, zinc oxide, silica, lead oxide, diatomaceous earth materials, colloidal materials, and microcrystalline synthetic polymers.

An assay device for the present invention can have many configurations, some of which are specifically illustrated herein. One embodiment of an instrumented diagnostic device 10 having an analytical chemistry strip or transport matrix of the present invention is illustrated in FIGS. 1 and 2. The device 10 includes a housing 12 having a receptor such as an inlet port 14 which extends from the surface 16 of the housing to its interior 18 for receiving a sample 20 containing the one or more analytes to be determined. The inlet port 14 allows the sample 20 to be introduced to a first 22 and second transport matrix 24 containing chemical reagents for determining the presence of one or more selected analytes in the sample 20.

Once the sample 20 is introduced to both the first 22 and second 24 transport matrices through the inlet port 14, the sample 20 is chemically reacted with at least one reagent on each of the transport matrices 22, 24 to produce a reaction product mixture corresponding to the reagent. A portion of the reaction product mixture is transported to at least one detection zone on each of the transport matrices 22, 24 and produces a physically detectable change which correlates with the amount of the corresponding selected analyte in the sample 20.

As specifically illustrated in FIG. 1, each of the first 22 and second 24 transport matrices contains two detection zones 26, 28 and 30, 32 respectively. Detectors 34 are positioned to measure optical radiation reflected from the detection zones 26, 28 on the first transport matrix. Detectors 36 are positioned to measure optical radiation reflected from the detection zones 30, 32 on the second transport matrix. The quality control zone 42 does not exhibit the physically detectable change measured in each of the detection zones. Each of the detection zones and the quality control zone are examples of different types of sampling areas on the transport matrices where reflected optical radiation is sampled and measured by one of the detectors.

A light-emitting diode (LED) 44 provides a source of optical radiation which is directed to each detection zone 26, 28 and 30, 32 and the quality control zone 42 by a plurality of totally internal-reflecting elements (TIR) 46 which act as mirrors and as a consequence of the refractive index of the transparent material from which they are formed, require no reflective coating.

The illumination from the LED 44 is split four ways. A part of the illumination is directed to the reference detector 40 from the reflecting element 48. Another part of the illumination is directed to detection zones 26, 28 from a series of reflecting elements 50, 52. The illumination is also directed to detection zones 30, 32 from a series of reflecting elements 54, 56. The reflecting element 46 illuminates another sampling area on the second assay strip 24 for a quality control detector 38.

FIG. 2 specifically illustrates another view of the device with an optics assembly 58 and printed circuit board (PCB) 60 disposed within the interior 18 of the housing. The inlet port 14 leads to the first 22 and second 24 assay strips which are supported on the optics assembly 58. Each of the detectors 34, 36, 38, 40 and the LED 44 are mounted directly to the PCB 60. A liquid crystal display (LCD) 62 is also located on the PCB 60 and is positioned to direct its display through a window 64 or opening in the exterior of the housing 12. The LED 44, each of the detectors 36, and the LCD 62 are connected through the PCB 60. A pocket of desiccant 66 can be provided to prevent moisture from affecting the shelf life stability or the operation of the device 10.

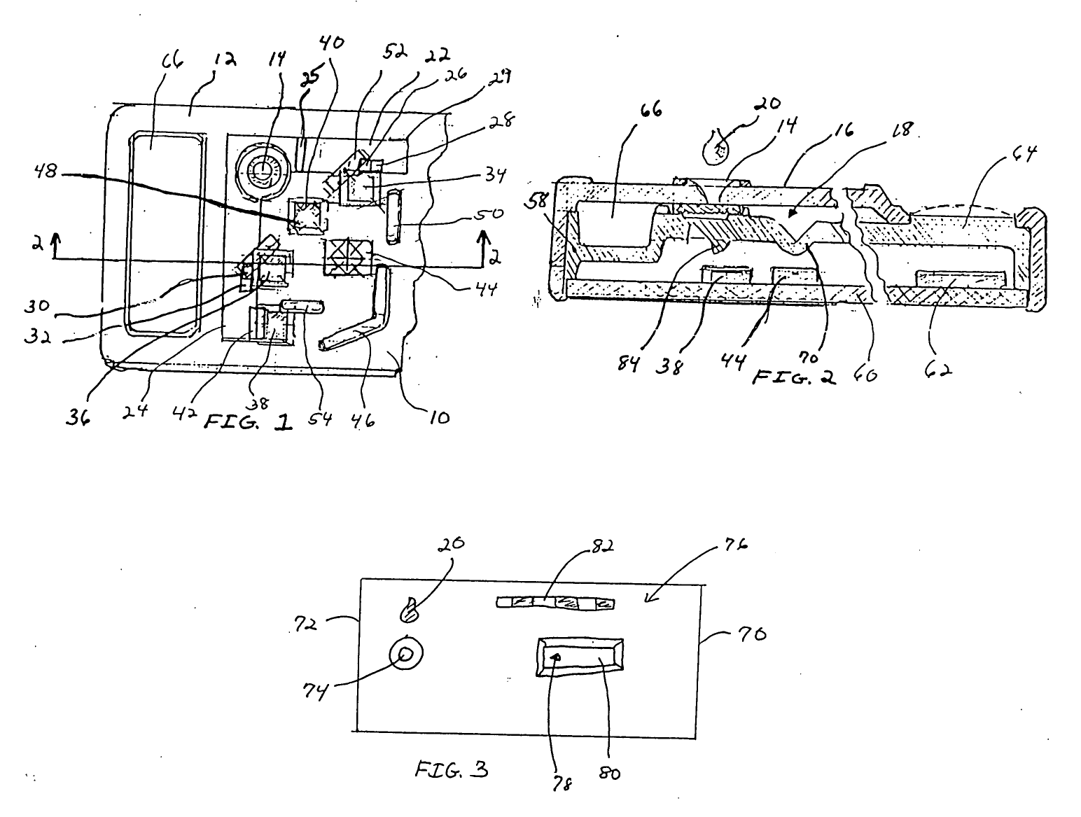

One embodiment of a non-instrumented diagnostic device 70 having an analytical chemistry strip or transport matrix of the present invention is illustrated in FIG. 3. The device 70 includes a housing 72 having a receptor such as an inlet port 74 which extends from the surface 76 of the housing to its interior for receiving the sample 20 containing one or more analytes to be determined. The inlet port 74 allows the sample 20 to be introduced to an assay strip 78 containing chemical reagents for determining the presence of one or more selected analytes in the sample 20.

Once the sample 20 is introduced to the assay strip 78 through the inlet port 74, the sample 20 is chemically reacted with at least one reagent on the assay strip 78 to produce a reaction product mixture corresponding to the reagent. A portion of the reaction product mixture is transported to at least one detection zone 80 on the assay strip and produces a physically detectable change which correlates with the amount of the corresponding selected analyte in the sample 20. The resulting color in the detection zone 80 can then be compared to a color bar 82 or other reference to visually determine the presence and concentration of the selected analyte. The term detection zone shall mean the area measured, by either visual observation or by instruments, for the physically detectable change.

Although the chemistry and configurations of the present invention may be used in an integrated assay device, the present invention can be used in any other instrumented reflectance or transmission meter as a replaceable reagent. Thus, the present invention also encompasses integrated assay instruments and analytical assay instruments comprising the present assay device.

Substantially all types of assays can be carried out with the present invention for a wide variety of analytes. Assays that can be performed include, but are not limited to, general chemistry assays and immunoassays. Both endpoint and reaction rate type assays can be accomplished with the present invention.

Single or multiple assays can be done at one time. For example, a single assay can be performed measuring cholesterol or one device can be set up to measure both total and HDL cholesterol from a single sample. One test device can be set up to measure one, two, three, or more analytes at one time.

Analyte, as used herein, is the substance to be detected which may be present in the test sample. For example, general chemistry assays can be performed for analytes such as, but not limited to, glucose, cholesterol, HDL cholesterol, LDL cholesterol, triglycerides, and BUN. For immunoassays, the analyte can be any substance for which there exists a naturally occurring specific binding member (such as, an antibody), or for which a specific binding member can be prepared. Thus, an analyte is a substance that can bind to one or more specific binding members in an assay. Analyte also includes any antigenic substances, haptens, antibodies, macromolecules, and combinations thereof. As a member of a specific binding pair, the analyte can be detected by means of naturally occurring specific binding partners (pairs) such as the use of intrinsic factor protein as a member of a specific binding pair for the determination of Vitamin B12, or the use of lectin as a member of a specific binding pair for the determination of a carbohydrate. The analyte can include a protein, a peptide, an amino acid, a hormone, a steroid, a vitamin, a drug including those administered for therapeutic purposes as well as those administered for illicit purposes, a bacterium, a virus, and metabolites of or antibodies to any of the above substances. In particular, such analytes include, but are not intended to be limited to, ferritin; creatinine kinase MB (CK-MB); digoxin; phenytoin; phenobarbital; carbamazepine; vancomycin; gentamicin, theophylline; valproic acid; quinidine; luteinizing hormone (LH); follicle stimulating hormone (FSH); estradiol, progesterone; IgE antibodies; vitamin B2 micro-globulin; glycated hemoglobin (Gly Hb); cortisol; digitoxin; N-acetylprocainamide (NAPA); procainamide; antibodies to rubella, such as rubella-IgG and rubella-IgM; antibodies to toxoplasma, such as toxoplasmosis IgG (Toxo-IgG) and toxoplasmosis IgM (Toxo-IgM); testosterone; salicylates; acetaminophen; hepatitis B core antigen, such as anti-hepatitis B core antigen IgG and IgM (Anti-HBC); human immune deficiency virus 1 and 2 (HIV 1 and 2); human T-cell leukemia virus 1 and 2 (HTLV); hepatitis B antigen (HBAg); antibodies to hepatitis B antigen (Anti-HB); thyroid stimulating hormone (TSH); thyroxine (T4); total triiodothyronine (Total T3); free triiodothyronine (Free T3); carcinoembryonic antigen (CEA); and alpha fetal protein (AFP). Drugs of abuse and controlled substances include, but are not intended to be limited to, amphetamine; methamphetamine; barbiturates such as amobarbital, secobarbital, pentobarbital, phenobarbital, and barbital; benzodiazepines such as librium and valium; cannabinoids such as hashish and marijuana; cocaine; fentanyl; LSD; methaqualone; opiates such as heroin, morphine, codeine, hydromorphone, hydrocodone, methadone, oxycodone, oxymorphone, and opium; phencyclidine; and propoxyphene. The details for the preparation of such antibodies and their suitability for use as specific binding members are well known to those skilled in the art.

The sample to be tested by the present invention for the presence of an analyte can be derived from any biological source, such as a physiological fluid, including whole blood or whole blood components including red blood cells, white blood cells, platelets, serum and plasma; ascites; urine; sweat; milk; synovial fluid; peritoneal fluid; amniotic fluid; cerebrospinal fluid; and other constituents of the body which may contain the analyte of interest. The test sample can be pre-treated prior to use, such as preparing plasma from blood, diluting viscous fluids, or the like; methods of treatment can involve filtration, distillation, concentration, and the addition of reagents. Besides physiological fluids, other liquid samples can be used such as water, food products and the like for the performance of environmental or food production assays. In addition, a solid material suspected of containing the analyte can be used as the test sample. In some instances it may be beneficial to modify a solid test sample to form a liquid medium or to release the analyte. The analyte can be any compound or composition to be detected or measured and which has at least one epitope or binding site.

For immunoassays, the present invention preferably uses particle detection for a physically detectable change or detectable response in each test zone related to the level of analyte in the sample. Other means for providing a physically detectable change in the test zones are suitable for use in the present invention. For example, and not for limitation, the analyte may be labeled with an indicator to measure electrical conductance or the reflectance or absorption of a characteristic light wavelength. The analyte may also be reacted with other chemicals to convert a dye, chromogenic compound or the like into a colored form detectable by means of transmission or reflectance photometry. As used herein, the terms signal producing reagent and indicator are meant to include all compounds capable of labeling the analyte or conjugate thereof and generating a detectable response or signal indicative of the level of analyte in the sample.

Another embodiment of the present invention non-diffusively immobilizes a chemical reagent on a solid phase support or a transport matrix which provides a zone in the path through which the sample flows. The transport matrix can be any solid material to which a chemical reagent can be immobilized and includes, but is not intended to be limited to, beads, magnetic particles, paramagnetic particles, microparticles or macroparticles, slides made of glass or other transparent material, capillary and test tubes, fabric or mesh that is woven or cast, and microtiter plates. The transport matrix can be made from synthetic materials, naturally occurring materials, or naturally occurring materials which have been synthetically modified, and includes, but is not intended to be limited to, cellulose materials, such as paper, cellulose and cellulose derivatives such as cellulose acetate and nitrocellulose; fiberglass; naturally occurring cloth such as cotton; synthetic cloth such as nylon; porous gels, such as silica, agarose dextran, and gelatin; porous fibrous matrices; starch based materials, such as cross-linked dextran chains; ceramic materials; olefin or thermoplastic materials including polyvinyl chloride, polyethylene, polyvinyl acetate, polyamide, polycarbonate, polystyrene, coploymers of vinyl acetate and vinyl chloride, combinations of polyvinyl chloride-silica; and the like. Often the transport matrix is a porous material or wicking member. By the term porous is meant that the material is one through which the test sample can easily pass and which supports the chemical reagent for exposure to the test sample.

Preferably, the transport matrix is used to transport the sample across an assay test zone for non-instrumented or instrumented assays to produce qualitative or quantitative results. Referring to FIG. 4, a preferred embodiment of the present invention provides the lateral flow assay strip 22 from FIG. 1 which includes three zones of which two detection zones 26 and 28 are test zones and one of the test zones is a reference zone. A first zone 84 treats the sample with a chemical reagent. The first detection zone 26 produces a signal with intensity inversely proportional to analyte concentration and the second detection zone 28 acts as a reference and produces a signal that is directly proportional to analyte concentration. The sum of the signals from the first and second detection zones 26, 28 is substantially equal at all analyte concentrations. Quantitative or qualitative results are achieved by instrumental reading of color intensity on the first detection zone 26, the second detection zone 28 or both the first and second detection zones 26, 28. The results expressed by any one detection zone can also be determined as a proportion of the sum of the actual results expressed by both detection zones. Quality reference is achieved by instrumental reading of both detection zones, the sum of which should be substantially constant within a specified range.

Both detection zones 26 and 28 contain an opacifier 86 which is preferably uniformly distributed both across the cross sectional area 88 and the depth profile 90 of the matrix 22 within the detection zones 26, 28. The opacifier 86 can be distributed in one or more of the detection zones depending on whether the resolution of the particular zone requires optimization beyond its initial reflectance. The term baseline reflectance signal defines the reflectance of the assay composition alone, or in a system, without the opacifier. The system can include the assay composition in combination with the components of the transport matrix and device described herein.

In a preferred embodiment, the transport matrix configuration may be of any dimension which provide the desired number of zones and which permit (a) the desired binding reactions to be completed in a reproducible manner and (b) detection of the physically detectable change or the reaction indicator to occur. Preferably, the present transport matrix is a total of no more than about 100 mm in length and about 6 mm wide, and more preferably, from about 10 mm to about 40 mm in length and about 1 mm to about 5 mm wide. The transport matrix is advantageously integrated into any reflectance based instrument, and more preferably, into a disposable electronic assay device, such as that described in above related applications, previously incorporated by reference. Although the chemistry and configurations of the present invention may be used in an integrated assay device, the present invention can be used in any other instrumented reflectance or transmission meter as a replaceable reagent.

The transport matrix can comprise a plurality of zones along its length. The zones can contain diffusively or non-diffusively bound reagents. Each zone can be from about 0.1 mm to about 10 mm wide, more preferably from about 0.25 mm to about 5 mm wide. There will be a minimum of two zones and a maximum of about 10 or more zones, depending on the number of assays to be conducted on one transport matrix.

The transport matrix can be one continuous section of bibulous material or can be composed of one, two, three or more sections. Each zone may be a separate bibulous material where each zone is in fluid communication with adjacent zones, or two or more adjacent zones may share a common material, with the other zones being different materials. The transport matrix including each of the zones can be composed of the same or different bibulous materials. The bibulous material permits fluid communication between the various zones, spacers (if present) and sample application site by wicking or capillary action upon application of a fluid sample.

In the preferred embodiments, the transport matrix includes a bibulous substrate to which the chemical reagent, which may be labeled, is diffusively or non-diffusively immobilized. Non-diffusive immobilization can be conducted by adsorbing, absorbing, crosslinking or covalently attaching the capture reagent to the bibulous substrate.

Diffusive immobilization can be conducted by formulating the assay reagent(s) to be immobilized (e.g., by dissolving in a suitable solvent such as water, a C₁-C₄ alcohol or mixture thereof, along with any desired additives), applying the resulting formulation to the bibulous material of the membrane, filter or transport layer in the desired location(s), and drying the material. Suitable additives may include detergents, proteins, blocking agents, polymers, sugars or the like. Alternatively, the additive(s) and assay reagent(s) may be applied to the membrane, filter or transport layer by precoating with a “blocking agent”, water soluble polymer, sugar or detergent, followed by depositing the conjugate or conjugate formulation and drying the material. Diffusive immobilization allows rapid reconstitution and movement of reagents, whether reacted or unreacted, through the bibulous substrate. Non-diffusive immobilization can be accomplished by covalently attaching, adsorbing or absorbing the capture reagent to the transport matrix. The zones can contain reagents diffusively or non-diffusively bound including, but not limited to, antibodies, antigens, enzymes, substrates, small molecules, proteins, recombinant proteins, viral or bacterial lysate, receptors, sugars, carbohydrates, polymers like PVA and detergents.

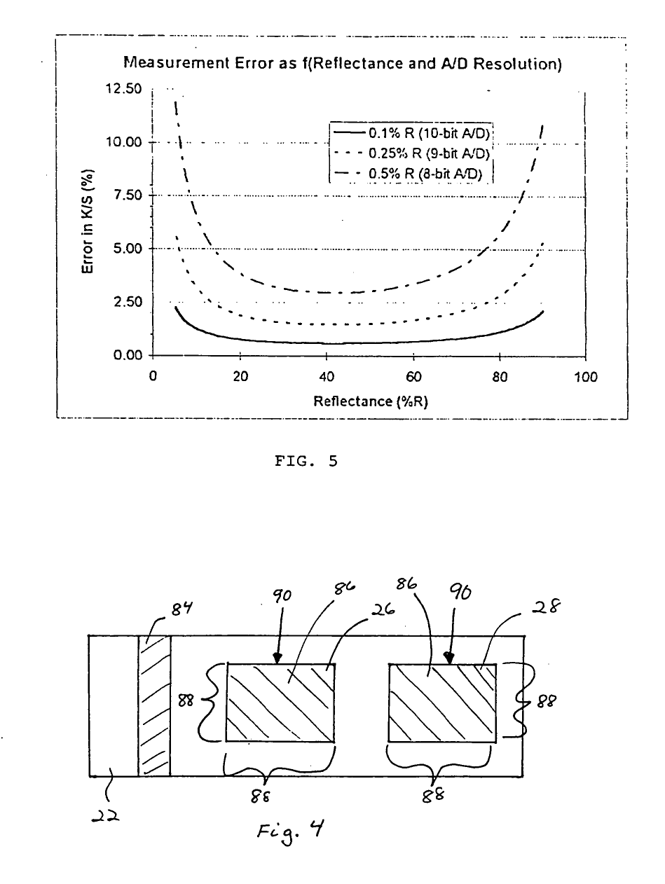

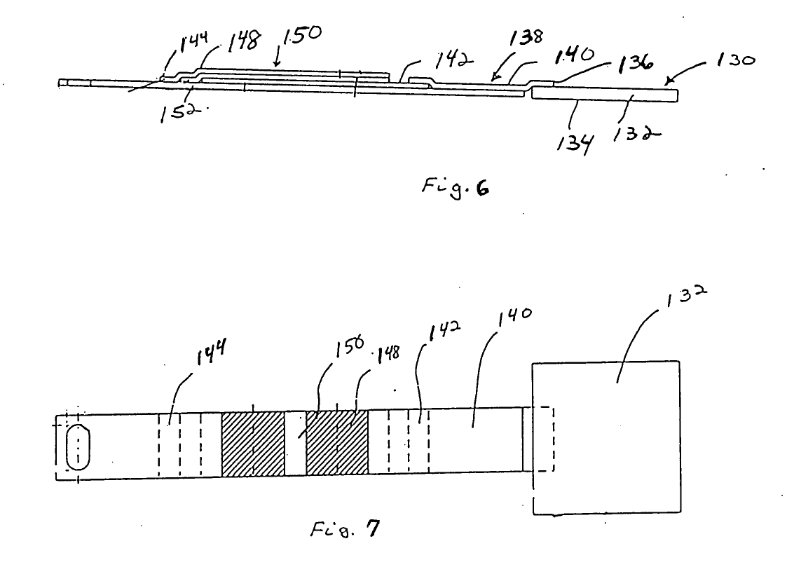

Adjusting the concentration of the opacifier is to place the analyte range in the region of most intense clinical interest or greatest resolution, requiring the greatest precision or lowest standard deviation of the error curve as illustrated in FIG. 5. This usually means between 20% and 80% reflectance, but can mean as low as 10% reflectance if the electronics of the reflectometer are of high quality and do not contribute a large amount of noise. By maximizing the signal-to-noise ratio (S/N), the signal range or “curve separation”, has only a linear affect on S/N. By contrast, noise has a quadratic affect because the noise component is squared. The lower the signal as represented by a lower reflectance percentage (% R) in FIG. 5, the greater the noise that will be relatively contributed by many components, such as analog-to-digital conversion. As used herein, the reflectance percentages are the percentages of relative reflectance. Whereas, absolute reflectance is defined as intensity of the reflected light over the intensity of incident light.

Addition of an opacifier will move the dose response curve to higher values of % R in FIG. 5. In this way, the colors produced by high (or, in some cases, low) concentrations of analyte can be adjusted to be within a more favorable range for an optimal S/N for any given reflectance instrument.

The method of determining a desired range of concentration for the opacifier to optimize the resolution of the assay includes formulating a series of membranes with varying opacifier concentrations and with the desired chemical reagent for the analysis of interest. A dose response experiment yields reflectance values over the desired range of analyte. If the reflectance results are at either the high (>80%) or low (<20%) end of the reflectance scale, then the optimal concentration of titanium dioxide is one that restores the balanced distribution of reflectance values.

If the reflectance results are beyond the high end of the reflectance scale, the present invention provides for decreasing the baseline reflectance signal for the assay composition. The baseline reflectance signal is optimized for the assay composition to produce at least the minimal level of reflectance decrease for the lowest analyte concentration expected to assay in the sample. This step is done prior to increasing the reflectance of optical radiation detecting a physical change of the sample-exposed assay composition to be within the range of optimal assay resolution. In the contrary example, wherein the chemical reagent system increases reflectance with an increasing analyte concentration, the baseline reflectance signal is decreased for the assay composition to produce no more than the minimal level of reflectance increase for the lowest analyte concentration expected to assay in the sample.

There are several methods for optimizing the baseline reflectance signal which include, but are not limited to increasing the porosity of the assay composition or the transport matrix which supports the assay composition. Increasing the pore size or fluid uptake/surface area of the support or transport matrix usually decreases its reflectance of optical radiation.

Another method of optimizing the baseline reflectance signal includes increasing the intensity of the detectable color change for a given concentration of analyte expected to assay in the sample. An example of increasing the color intensity is to add more of the indicator color species or label.

Another method of optimizing the baseline reflectance signal includes adjusting the performance of the electronic components of a reflectometer, if an instrument is used to detect the physical change. Usually, a decrease of the baseline reflectance signal will result in a decrease in the signal to noise ratio. In the preferred embodiment which uses a reflectometer, the signal to noise ratio of a reflectometer is determined prior to the step of increasing the reflectance. The signal to noise ratio of the reflectometer is adjusted over a predetermined range of reflectance. The opacifier is then impregnated in the assay composition in an amount sufficient to increase the reflectance of optical radiation detecting a physical change of the sample exposed assay composition to be within the range of optimal assay resolution.

The present invention provides a method for determining the level of a selected analyte in a sample. The method includes optimizing a physically detectable change in an assay composition which correlates with the amount of selected analyte when contacted with the sample. Preferably, the assay composition is supported in a detection zone on a transport matrix and the opacifier is uniformly distributed across the two dimensional plane forming the cross-sectional area of the detection zone. The opacifier is also distributed at least partially across the depth profile of the matrix in the detection zone.

Preferably, the opacifier is distributed by contacting the assay composition or matrix with a solution containing the opacifier to impregnate the opacifier into at least a portion of the depth profile of the assay composition or matrix. Another example of distributing the opacifier is to spray the assay composition or matrix with a solution containing the opacifier to achieve the desired impregnation.

The transport matrices shown and described herein can be configured by several assembly methods. Conventional methods of immobilizing a reagent, by dipping a transport matrix in a solution containing the reagent and subsequently drying are suitable for use in portions of the present invention. In a hybrid method, an approximately uniform layer or coating of a reagent is first deposited on the transport matrix. The uniform deposit is then modified by deposited a pattern of a second reagent over the first reagent or in a zone on the transport matrix prior to the first reagent. The following examples specify the details of these methods. These methods can also be used for impregnating the described assay compositions.

Having generally described the present invention, a further understanding can be obtained by reference to the following specific examples, which are provided herein for purposes of illustration only and are not intended to be limiting of the present invention.

### EXAMPLE 1

The following strip assembly and chemical reagent immobilization methods were used in the construction of the inventive examples. A dipping mixture of 400 mL of 15% (w/v) titanium dioxide was prepared for the creatinine reagent strip by mixing about 60 g of titanium dioxide (Kronos 2020—Titanium Dioxide, Rutile, purchased from Kronos, Inc., Houston, Tex.) and about 0.3 g. silicon dioxide (Aerosil 200—Silicon Dioxide, amorphous fumed silica, obtained from the Degussa Corporation, Ridgefield Park, N.J.) in a container. About 0.3 g of sodium tripolyphosphate obtained from Sigma Chemical Co., St. Louis, Mo. was weighed and added along with the other dry agents to the same mixing container. Using a metal spatula, the dry agents were carefully stirred until evenly mixed.

From a separate container, about 300 mL of 1% (w/v) PVA 186K, which is a 1% (w/v) solution of Poly(vinyl alcohol), 87-89% hydrolyzed, average MW 186,000 obtained from the Aldrich Chemical Company, Inc., Milwaukee, Wis., was slowly added to the dry components while using a high speed mixer set at the lowest setting. After adding all the dry components to the PVA solution, the mixer speed was slowly increased to the highest setting and stirred at this setting for 5 minutes. About 0.8 mL of BYK-024 which is poly(propylene glycol), from BYK-Chemie USA, Wallingford, Conn., was slowly pipetted into the solution with mixing at the highest setting to remove foam. After removing the mixing blade, the titanium dioxide mixture was transferred into a 500 mL graduated cylinder. The volume was increased to about 400 mL with 1% PVA 186K. The titanium dioxide mixture was transferred into a 1 L glass Erlenmeyer flask containing a magnetic stir bar and stirred for about an hour prior to use. This mixture was kept at room temperature, but was stirred for at least 1 hour or until homogenous before use.

Other mixtures were prepared in accordance with this procedure by varying the concentration of the titanium dioxide. For example, additional mixtures containing 5%, 10%, and 20% (w/v) titanium dioxide were also prepared.

FIGS. 6 and 7 illustrate a laminated strip layout 130 for a sarcosine, creatinine or other general chemistry assay that is suitable for use in the preferred embodiment of the diagnostic device described above. The strip layout 130 includes a sample pad 132 for receiving the sample through the inlet port (not shown) on the topside 134 of the pad 132 at the proximal end 136 of the strip 138.

The sample pad 132 is made of either CytoSep No. 1660 or GF/QA from Whatman. The GF/QA material from Whatman, Inc. of Fairfield, N.J. which is an quaternary ammonium cellulose matrix having a basis weight of about 68 g/m², a thickness of about 373 μm, and a mean pore size of 4.0 μm. The GF/QA material has a protein binding capacity for bovine serum albumin of 0.296 g/dg with a linear wicking (Klemm) of 2 min for a 7.5 cm rise and a derivative content of 2.0 mg/cm². The GF/QA material includes trimethylhydroxy propyl quaternary ammonium (QA) as a high performance strong base quaternary ammonium exchanger with fast kinetics, high protein capacity, and is effective over a wide pH range. The material had approximately square dimensions of about 7 mm with a thickness of about 0.023 inches.

The sample flows from the sample pad 132 to a sample treatment pad 140 that is made of a material from Pall Biosupport Accuwik No. 14-20, is about 7 mm long and 3 mm wide with a thickness of about 0.00945 inches. The sample treatment pad 140 is in fluid communication with a transport matrix 142 made of polyester substrate from Tetko P/N 7-2F777 BM having a size of about 11 mm long and about 3 mm wide with a thickness of about 0.00846 inches. The transport matrix 142 allows the treated sample to flow quickly towards the distal end 144 of the strip. Substantially overlapping the transport matrix 142 is a spreading layer 146 that assists in spreading the treated sample across the length of the strip. A reagent layer 148 substantially overlaps the spreading layer 146 and contains the chemical reagents for performing the assay to produce a physically detectable change on the top surface 150 of the reagent layer that is measured by the detector previously described. The reagent layer contains the dried chemical components needed to measure sarcosine in the sample: the solution for dipping the indicator included 0.5% w/v sucrose, 1.0% w/v polyvinyl-pyrrolidone (avg. mw. about 40,000), 5% v/v surfactant 10G (p-isononylphenoxypoly(glycidol)) and 75 mg/ml bis(4-(N-(3′-sulfo-n-propyl)-N-n-propyl)amino-2,6-dimethyl-phenyl)methane, disodium salt; the enzyme solution used for dipping the reagent layer included 1000 u/ml horse radish peroxidase (EC 1.11.17), 500 u/ml sarcosine oxidase (EC 1.5.3.1), (all from the Toyobo Company), 1% w/v poly(vinyl alcohol) (avg. mw. about 70,000), 1% v/v Triton X-100 (t-octylphenoxypolyethoxyethanol), 1% w/v sucrose, 5 mg/ml Bovine Serum Albumin, and 50 mM buffer 3-(N-morpholino)-2-hydroxypropanesulfonic acid, sodium salt, pH 7.5.

The transport matrix 142 was dipped in the titanium oxide solution described above for several minutes and allowed to air dry. The sample treatment pad 140 and the transport matrix 142 were supported and attached to a backing material 152 made of poly(ethylene terephthalate) plastic from Adhesives Research with an adhesive P/N 8565. The backing material was about 22.5 mm long and about 3 mm wide with a thickness of about 0.01 mm.

FIG. 8 presents assay results for various concentrations of sarcosine, namely about 2 mM, 3 mM, 5 mM, 15 mM, and 30 mM, using transport matrices impregnated with different concentrations of titanium dioxide, namely 5%, 10%, 15%, and 20% (w/v) titanium dioxide. The assay results in FIG. 8 are presented in units of K/S calculated using the equation K/S=(1-R)²/2R; wherein R is reflectance (R=% R/100); from Kubelka and Munk, Z. Techn. Phys., 12, 593-601 (1933) and explained in Werner and Rittersdorf, Methods of Enzymatic Analysis, Vol. I, pp. 305-326 (Verlag Chemie, 1983). The reflectance densities presented in FIG. 8 was measured with a hand-held reflectance densitometer made by Gretag which uses about a one mm diameter measurement area.

As illustrated in FIG. 8, the reflectance percentage was increased by increasing the concentration of titanium dioxide, particularly with increasingly higher concentrations of sarcosine. The increase in reflectance percentage demonstrates a means of modulating the color response of the assay by impregnating scattering centers into the transport matrix.

### EXAMPLE 2

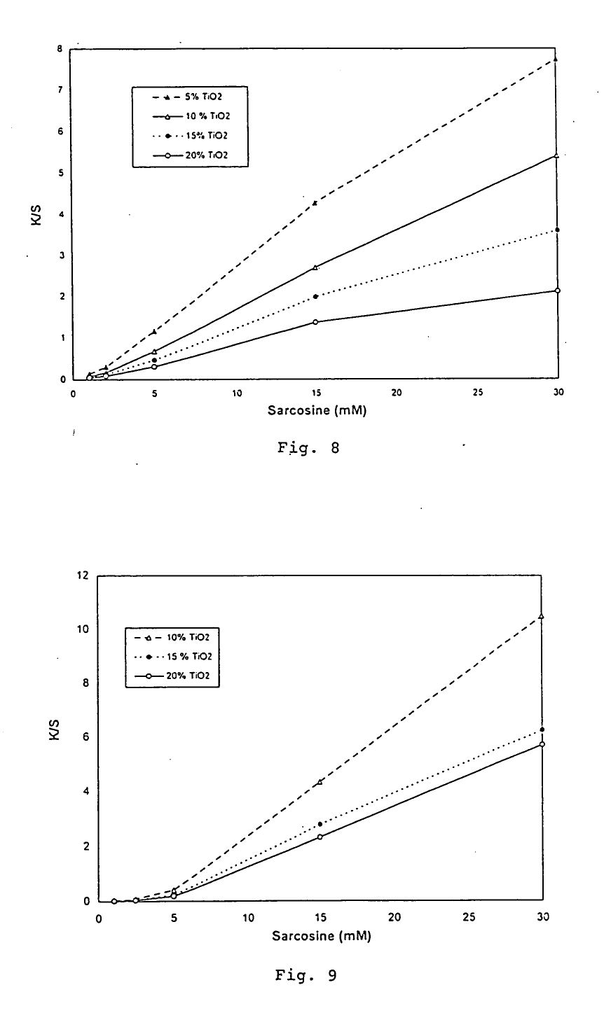

Additional assays were prepared in a manner similar to that described in Example 1. The only difference was the substitution of N-ethyl-N-(2-hydroxy-3-sulfo-propyl)-3,5-dimethylaniline, sodium salt, monohydrate, commercially available under the tradename MAOS from Dojindo, for bis(4-(N-(3′-sulfo-n-propyl)-N-n-propyl)amino-2,6-dimethyl-phenyl)methane, in the solution for dipping the indicator. FIG. 9 presents assay results for various concentrations of sarcosine, namely about 2 mM, 3 mM, 5 mM, 15 mM, and 30 mM, using transport matrices impregnated with different concentrations of titanium dioxide, namely 10%, 15%, and 20% (w/v) titanium dioxide. The assay results in FIG. 9 were measured and calculated in the same manner as in FIG. 9.

Numerous modifications and variations of the present invention are possible in light of the above teachings. It is therefore to be understood that within the scope of the appended claims, the invention may be practiced otherwise than as specifically described herein.

1. An assay composition for producing a physically detectable change upon contact with a sample which correlates with the amount of selected analyte in the sample, the composition comprising:
a support matrix pervious to optical radiation;
a chemical reagent yielding a physically detectable change which correlates with the amount of selected analyte in the sample, the support matrix having at least one detection zone for detecting the physical change, the detection zone having a cross-sectional area and a depth profile extending into the support matrix;
an opacifier present in an amount sufficient to increase the resolution of the physically detectable change, the opacifier being distributed uniformly across the cross-sectional area of the detection zone and being distributed through at least a portion of the depth profile of the support matrix within the detection zone, the chemical reagent being substantially immobilized relative to the opacifier when detecting the physical change.

2. The composition of claim 1 wherein the opacifier is present in an amount sufficient to increase the reflectance of optical radiation into a range providing the least error for detecting the physical change.

3. The composition of claim 1 wherein the opacifier is present in an amount sufficient to increase the reflectance of optical radiation into a range providing the greatest contrast for detecting the physical change with the smallest change in the selected analyte concentration.

4. The composition of claim 1 wherein the opacifier is present in an amount sufficient to increase the reflectance of optical radiation into a range of about 20% to about 80% reflectance percentage which minimizes the error in K/S for the analytical range of clinical interest in the selected analyte.

5. The composition of claim 1 wherein the physically detectable change is a variation of color.

6. The composition of claim 1 wherein the opacifier is distributed uniformly through the depth profile of the support matrix within the detection zone.

7. The composition of claim 1 wherein the opacifier is present in an amount of about 5% to about 15% by weight/volume of the opacifier.

8. The composition of claim 1 wherein the opacifier is present in an amount of about 10% to about 15% by weight/volume of the opacifier.

9. The composition of claim 1 wherein the opacifier is selected from the group consisting of titanium dioxide, barium sulfate, zinc oxide, silica, lead oxide, diatomaceous earth materials, colloidal materials, and microcrystalline synthetic polymers.

10. The composition of claim 1 wherein the opacifier is rutile titanium dioxide.

11. The composition of claim 1 wherein the opacifier having a surface area in the amount greater than about 10 m²/g.

12. The composition of claim 1 wherein the opacifier is in the shape of particles, each particle having a diameter in the range of about 0.2 μm to about 0.4 μm.

13. The composition of claim 1 wherein the opacifier is in the shape of particles, each particle having a diameter of about 0.3 μm.

14. A transport matrix producing a physically detectable change in a detection zone which correlates with the amount of selected analyte in a sample, the matrix comprising:
a detection zone having a chemical reagent yielding a physically detectable change which correlates with the amount of selected analyte in the sample, the detection zone having a cross-sectional area and a depth profile extending into the matrix;
an opacifier present in an amount sufficient to increase the resolution of detecting the physical change, the opacifier being distributed uniformly across the cross-sectional area of the detection zone and being distributed through at least a portion of the depth profile of the matrix within the detection zone, the opacifier being substantially immobilized relative to the chemical reagent in the detection zone when detecting the physical change.

15. The transport matrix of claim 14 wherein the opacifier is present in an amount sufficient to increase the reflectance of optical radiation into a range providing the least error for detecting the physical change.

16. The transport matrix of claim 14 wherein the opacifier is present in an amount sufficient to increase the reflectance of optical radiation into a range providing the greatest contrast for detecting the physical change with the smallest change in the selected analyte concentration.

17. The transport matrix of claim 14 wherein the opacifier is present in an amount sufficient to increase the reflectance of optical radiation into a range of about 20% to about 80% reflectance percentage which minimizes the error in K/S for the analytical range of clinical interest in the selected analyte.

18. The transport matrix of claim 14 wherein the physically detectable change is a variation of color.

19. The transport matrix of claim 14 wherein the opacifier is distributed uniformly through the depth profile of the matrix within the detection zone.

20. The transport matrix of claim 14 wherein the opacifier is present in an amount of about 5% to about 15% by weight/volume of the opacifier.

21. The transport matrix of claim 14 wherein the opacifier is present in an amount of about 10% to about 15% by weight/volume of the opacifier.

22. The transport matrix of claim 14 wherein the opacifier is selected from the group consisting of titanium dioxide, barium sulfate, zinc oxide, silica, lead oxide, diatomaceous earth materials, colloidal materials, and microcrystalline synthetic polymers.

23. The transport matrix of claim 14 wherein the opacifier is rutile titanium dioxide.

24. The transport matrix of claim 14 wherein the opacifier having a surface area in the amount greater than of about 10 m²/g.

25. The transport matrix of claim 14 wherein the opacifier is in the shape of particle having a size in the range of about 0.2 μm to about 0.4 μm.

26. The transport matrix of claim 14 wherein the opacifier is in the shape of particle having a size of about 0.3 μm.

27. A diagnostic device for determining the presence of a selected analyte in a sample, the device comprising:
a housing having an exterior surface and sealing an interior area;
a receptor configured to receive the sample containing an analyte selected for determining its presence, the receptor being located on the exterior surface of the housing;
at least one transport matrix for reacting the sample with a chemical reagent to yield a physically detectable change in a detection zone which correlates with the amount of selected analyte in the sample, the detection zone having a cross-sectional area and a depth profile extending into the matrix, an opacifier present in an amount sufficient to increase the resolution of detecting the physical change, the opacifier being distributed uniformly across the cross-sectional area of the detection zone and distributed through at least a portion of the depth profile of the matrix within the detection zone, the opacifier being substantially immobilized relative to the chemical reagent in the detection zone when detecting the physical change.

28. The device in claim 27 wherein the transport matrix produces a physically detectable change in a plurality of detection zones which correlates with the amount of selected analyte in a sample, the matrix including at least a first and a second detection zone, at least one of the detection zones having the chemical reagent yielding a physically detectable change which correlates with the amount of selected analyte in the sample, the opacifier being immobilized in the detection zone relative to the chemical reagent.

29. The device in claim 27 wherein the first and second detection zone contain the reagent and the opacifier.

30. The device in claim 27 wherein the selected analyte is creatinine.

31. The device of claim 27 wherein the opacifier is present in an amount sufficient to increase the reflectance of optical radiation into a range providing the least error for detecting the physical change.

32. The device of claim 27 wherein the opacifier is present in an amount sufficient to increase the reflectance of optical radiation into a range providing the greatest contrast for detecting the physical change with the smallest change in the selected analyte concentration.

33. The device of claim 27 wherein the opacifier is present in an amount sufficient to increase the reflectance of optical radiation into a range of about 20% to about 80% reflectance percentage which minimizes the error in K/S for the analytical range of clinical interest in the selected analyte.

34. The device of claim 27 wherein the opacifier is distributed uniformly through the depth profile of the matrix within the detection zone.

35. The device of claim 27 wherein the opacifier is selected from the group consisting of titanium dioxide, barium sulfate, zinc oxide, silica, lead oxide, diatomaceous earth materials, colloidal materials, and microcrystalline synthetic polymers.

36. A method for determining the level of a selected analyte in a sample, the method comprising the step of:
optimizing a physically detectable change in an assay composition which correlates with the amount of selected analyte when contacted with the sample.

37. The method of claim 36 wherein the optimizing step includes supporting the assay composition in a detection zone on a transport matrix.

38. The method of claim 36 wherein the optimizing step includes supporting a reagent and an opacifier in the detection zone and increasing the reflectance of the physically detectable change with the opacifier.

39. The method of claim 36 wherein the supporting step includes distributing the opacifier uniformly across the cross-sectional area of the detection zone and distributing the opacifier at least partially across the depth profile of the matrix in the detection zone.

40. The method of claim 36 wherein the supporting step includes distributing the opacifier uniformly through the depth profile of the matrix in the detection zone.

41. The method of claim 40 wherein the distributing step includes impregnating the opacifier by contacting the matrix with a solution containing the opacifier.

42. The method of claim 40 wherein the distributing step includes impregnating the opacifier by spraying the matrix with a solution containing the opacifier.

43. A method of optimizing the assay results of a selected analyte in a sample-exposed assay composition, the method comprises:
increasing the reflection of optical radiation detecting a physical change of the sample-exposed assay composition to be within the range of optimal assay resolution.

44. The method of claim 43 wherein the increasing step includes:
scattering the optical radiation directed within the sample-exposed assay composition prior to detecting the physical change of the sample-exposed assay composition.

45. The method of claim 43 wherein, prior to the step of increasing the reflectance, the method includes decreasing the baseline reflectance signal for the assay composition.

46. The method of claim 43 wherein, prior to the step of increasing the reflectance, the method includes optimizing the baseline reflectance signal for the assay composition to produce at least the minimal level of reflectance decrease for the lowest analyte concentration expected to assay in the sample.

47. The method of claim 46 wherein the step of optimizing the baseline reflectance signal includes increasing the porosity of the assay composition.

48. The method of claim 46 wherein the step of optimizing the baseline reflectance signal includes increasing the intensity of the detectable color change for a given concentration of analyte expected to assay in the sample.

49. The method of claim 43 wherein, prior to the step of increasing reflectance, the method includes:
determining the baseline reflectance signal of the assay composition;
impregnating an opacifier in the assay composition in an amount sufficient to increase the reflectance of optical radiation detecting a physical change of the sample exposed assay composition to be within the range of optimal assay resolution.

50. The method of claim 43 wherein the method includes supporting the assay composition in a detection zone on a transport matrix.

51. The method of claim 43 wherein, prior to the step of increasing reflectance, the method includes:
adjusting the signal to noise ratio of a reflectometer over a predetermined range of reflectance;
impregnating an opacifier in the assay composition in an amount sufficient to increase the reflectance of optical radiation detecting a physical change of the sample exposed assay composition to be within the range of optimal assay resolution; and
subsequent to the step of increasing the reflectance, detecting the physical change of the assay composition with a reflectometer.
